(19) United States
(12) Patent Application Publication
(10) Pub. No.: US 2017/0112403 A1
(43) Pub. Date: Apr. 27, 2017

(54) SPECTRAL DECOMPOSITION AND DISPLAY OF THREE-DIMENSIONAL ELECTRICAL ACTIVITY IN THE CEREBRAL CORTEX

(71) Applicant: Cerebral Diagnostics Canada Incorporated, Toronto (CA)

(72) Inventors: Mark S. Doidge, Toronto (CA); Joseph D. Mocanu, Toronto (CA)

(21) Appl. No.: 15/295,717

(22) Filed: Oct. 17, 2016

Related U.S. Application Data
(63) Continuation of application No. 13/504,678, filed on Aug. 17, 2012, now abandoned.
(60) Provisional application No. 61/255,120, filed on Oct. 27, 2009.

(51) Int. Cl. A61B 5/04; A61B 5/048; A61B 5/00

(52) U.S. Cl. CPC A61B 5/04014; A61B 5/742; A61B 5/4076; A61B 5/048; A61B 5/7257

(57) ABSTRACT

Systems and methods are provided for measuring electrical activity within a brain of a patient. An electrode array is configured to take measurements of electrical potential as raw electroencephalographic (EEG) data. A data processing component includes a spectral decomposition component configured to divide the raw EEG data into a plurality of frequency intervals, within a total range of frequencies and an inverse solution component configured to transform the raw EEG data associated with each frequency interval into a spatial mapping of electrical activity as to provide a set of parameters, with each parameter representing an average electrical activity at an associated location within the brain over an epoch of interest.

### RELATED APPLICATIONS

This application is a continuation of co-pending U.S. application Ser. No. 13/504,678, filed Aug. 10, 2013, and entitled “SPECTRAL DECOMPOSITION AND DISPLAY OF THREE-DIMENSIONAL ELECTRICAL ACTIVITY IN THE CEREBRAL CORTEX”, which is a national stage application from International Application No. PCT/IB2010/002973, filed Oct. 27, 2010, which in turn claims priority from U.S. Provisional Patent Application Ser. No. 61/255,120, filed on Oct. 27, 2009. The subject matter of each of these applications is incorporated herein by reference.

### BACKGROUND OF THE INVENTION

The field of neuroscience known as “functional localization” attempts to assign functions to specific regions of the brain. For example, the temporal lobe has been shown to be involved with hearing, and the occipital lobe is involved in vision. Functional localization can be performed using functional magnetic resonance imaging (fMRI) or positron emission tomography (PET), both of which are quite expensive. In addition, there are a number of neurological conditions in which the afflicted area has yet to be determined or is undeterminable using the aforementioned methods. Given the number of neurological conditions that currently rely on subjective means of diagnosis or expensive medical imaging, there is a definite need to isolate meaningful signals from the brain in an objective and cost efficient manner.

Many of the brain's higher functions including those associated with thought, action, emotion, and sensation are often prone to illness, and they have been found to originate within the cerebral cortex, the convoluted outer surface of the brain commonly known as ‘gray matter’. The cerebral cortex emits electromagnetic signals, which can be measured by electrodes placed on the surface of the scalp. This practice, when graphed as waveforms plotting electrical potential (voltage) against time is generally known as electroencephalography (EEG).

### SUMMARY OF THE INVENTION

In accordance with an aspect of the present invention, a system is provided for measuring electrical activity within a brain of a patient. An electrode array is configured to take measurements of electrical potential as raw electroencephalographic (EEG) data. A data processing component includes a spectral decomposition component configured to divide the raw EEG data into a plurality of frequency intervals, within a total range of frequencies and an inverse solution component configured to transform the raw EEG data associated with each frequency interval into a spatial mapping of electrical activity as to provide a set of parameters, with each parameter representing an average electrical activity at an associated location within the brain over an epoch of interest.

In accordance with another aspect of the present invention, a computer readable medium stores executable instructions for evaluating electrical activity within a brain of a patient. A spectral decomposition component is configured to divide raw EEG data into a plurality of frequency intervals. Each frequency interval represents a frequency interval within a total range of frequencies. An inverse solution component is configured to reconstruct the electrical activity of at least a portion of the brain from the EEG data associated with each frequency interval into a spatial mapping of electrical activity as to provide a set of parameters for each frequency interval. Each parameter represents an average electrical activity at an associated location within the brain over a period of time. A user interface is configured to provide the set of parameters associated with at least one frequency interval to an associated output device.

In accordance with yet another aspect of the present invention, a method is provided for the analysis of raw EEG data of a subject. Raw EEG data is generated at an electrode array. The raw EEG data is filtered to produce a plurality of frequency intervals, with each frequency interval representing data within an associated frequency interval of the raw EEG data. The data represented by at least one of the plurality of frequency intervals is transformed via an inverse solution approximation algorithm as to determine, for each of the at least one frequency interval, values at a plurality of locations within a brain of the subject. Each value represents a current density within the frequency interval associated with the frequency interval at a corresponding location. The values corresponding to the current density associated with each of the at least one frequency interval are averaged over an epoch to produce, for each frequency interval, a plurality of averaged values corresponding to the plurality of locations within the brain of the subject. A set of the plurality of averaged values are displayed at a corresponding display.

### BRIEF DESCRIPTION OF THE DRAWINGS

FIG. 1 depicts a flowchart of a method in accordance with the present invention;

FIG. 2 depicts a flowchart of one implementation of a method in accordance with an aspect of the invention;

FIGS. 3A-3D depicts examples of visualization methods available to view the decomposed data. (A) 2D representation of all average current density values (one square per value) for each Brodmann area (Y-axis) and for each frequency band (X-axis). (B) The average current densities of each Brodmann area for one frequency band of interest (12-16 Hz in this example), visualized in a 3-D surface view. (C) The same data as in FIG. 3B visualized as 2D tomographic slices in the axial, or transverse, plane. (D) Data represented in histogram form, where the X-axis represents average current density values for one frequency band of interest and the Y-axis labels the brain areas described by the data;

FIG. 4 illustrates a computer system that can be employed to implement the systems and methods described herein as computer executable instructions, stored on a computer readable medium and running on the computer system.

FIG. 5 illustrates one implementation of a system 600 in accordance with an aspect of the present invention; and

FIGS. 6A-6E depict a series of images illustrating an example of how to use the linear average functionality of the present system to find a summary of the generator activity of an EEG waveform.

### DETAILED DESCRIPTION OF THE INVENTION

In accordance with an aspect of the present invention, methods and systems are provided to allow a user to decompose, analyze and visualize the three dimensional (3-D) electrical activity within the cerebral cortex of the brain. The human cerebral cortex performs numerous functions, and there are numerous diseases for which the present invention may be utilized for diagnosing, monitoring, and treating.

The present invention may be used as an aid to diagnose, aid to monitor and aid to treat a number of mental and behavioral disorders involving electrical abnormalities in the cerebral cortex, including diseases of the nervous system, medical conditions and psychological conditions. In many disorders, there are known abnormalities evident within the EEG and/or further QEEG analyses, that is, quantitative EEG, an extension of conventional EEG involving topographic maps. The methods of the present invention are such that they are designed to isolate spectral bands, that is, specific frequencies or frequency ranges of EEG signals, vector temporal-spatial movement dynamics (changes in the direction and location of electrical flow) and to correlate the generator sources (putative sources of electrical activity within the cerebral cortex) in three dimensions, thus identifying the locations and cortical electrical dynamics that underlie the EEG abnormalities. Regarding other items on the list below there are theoretical grounds for placing them here. The present invention is particularly suited for characterizing the abnormal electrical dynamics of many diseases involving the cerebral cortex. For any condition that exhibits EEG abnormalities, it is possible to better characterize and localize them with the present invention. Diagnostic tests may be developed with the aid of clinical trials to create sets of normative data derived from normal individuals, and sets of data derived from diseased individuals, which will be used for comparisons with data from a patient.

The listing of potential applications herein includes not only the names of diseases and disorders, but also the names of categories of diseases and disorders, which have accepted definitions under the World Health Organization ICD1O system of nomenclature. This includes block F (Chapter V) of the ICD-1O.

Organic mental disorders include Alzheimer's, vascular dementia, organic amnesic syndromes, and other cortical dementias. There are examples in scientific literature showing electrical abnormalities in people with dementias. In Alzheimer's, increased low frequency activity (where activity is defined as higher EEG signal amplitude) and decreased mean frequency is often found within the EEG signals. In some dementias, the EEGs revealed increases in delta (1-3 Hz) and/or theta (4-7 Hz) power (where power is defined as the square of the EEG signal amplitude) and decreased mean frequency as well as decreased beta (12-35 Hz) power and dominant frequency in the occipital lobe. Since EEG has very little ability to localize these abnormalities, it is possible to better characterize them with the methods of the present invention as an aid to diagnosis, monitoring and treatment. Pick's Disease has EEG abnormalities, which can be imaged and quantified in greater detail with the methods and apparatus of the present invention. Delirium Tremens has high frequency abnormalities on EEG.

A method in accordance with the present invention examines current density data in numerous spectral bands of discrete frequencies, which adds a further layer of useful information on which analysis can be performed. It can also analyze brain signals as either activity over brief periods of time (referred to here as brain events, such as fleeting thoughts, feelings, beliefs, or sensations), and longer activities (which are referred to here as brain states). The spectrally decomposed volumetric data can be averaged into epochs of a desired sample length to summarize the data or obtain an overall brain event or brain-state of the cerebral cortex for a prescribed period of time, further facilitating analysis.

The visualization system disclosed herein can also be used to identify mental and behavioral disorders due to psychoactive substance use, specifically substance abuse and drug-induced states affecting the cortex. This includes the stimulatory/depressive, toxic and withdrawal effects of psychoactive drugs such as, but not limited to, depressants, sedatives, stimulants, illegal narcotics, anti-epileptics, anxiolytics, sleep drugs, anti-psychotics, hallucinogens, anti-depressants, and inhalants. Examples include, but are not limited to, cocaine, amphetamines, cannabis, caffeine, tobacco, nicotine, LSD, ecstasy, GHB, PCP, heroin, opium, hashish, mescaline, “magic mushrooms”, and alcohol. For example, studies of alcohol abuse have found increased beta activity, and alcohol intoxication studies have found decreased alpha activity and increased theta activity. Increased alpha activity in frontal regions is associated with cannabis withdrawal and intoxication. Increased alpha and decreased delta activity is associated with crack cocaine withdrawal. The present invention may be used to determine their effects on the electromagnetic activities of the cortex, which will be used to diagnose and plan the treatment of cortical states caused by these drugs.

Systems and methods in accordance with the present invention can also be used to diagnose schizophrenia, schizotypal disorders, and delusional disorders. For example, schizophrenics occasionally exhibit low mean alpha frequency as well as other alpha wave abnormalities or abnormalities of other frequency bands, including frontal delta and theta excess on EEG. Similarly, affective disorders include but not limited to unipolar and bipolar disorders including depression and mania. Alpha and theta wave abnormalities such as increased alpha and theta power are known to exist in unipolar depressed patients. Bipolar patients tend towards reduced alpha and beta activity.

Neurotic, stress-related, and somatoform disorders can also be detected via the disclosed visualization system. Neurotic, stress-related and somatoform disorders include but are not limited to anxiety disorders, obsessive-compulsive disorder, reaction to severe stress, dissociative disorders, and somatoform disorders. Anxiety disorders often have reduced alpha activity. Similarly, the system can facilitate diagnosis of behavioral syndromes associated with physiological disturbances and physical factors. This includes behavioral syndromes associated with physiological disturbances and physical factors including but not limited to anorexia nervosa, bulimia nervosa, non-organic sleep disorders including non-organic insomnia and non-organic hypersomnia, and non-organic disorder of the sleep-wake schedule, sleepwalking (somnambulism), sleep terrors, nightmares, and sexual dysfunction not caused by organic disorders or diseases.

The visualization system can also be used to diagnose disorders of adult personality and behavior as well as disorders of psychological and intellectual development. Disorders of adult personality and behavior can include, but are not limited to, paranoid, schizoid, dissocial, emotionally unstable, histrionic, anakastic, anxious, dependant personality disorders as was as personality disorder unspecified types, and habit and impulse disorders including pathological gambling, gender identity disorders, disorders of sexual preference, psychological and behavioral disorders associate with sexual development and orientation. Disorders of psychological development can include specific developmental disorders of speech and language, specific developmental disorders of scholastic skills including developmental dyslexia, specific developmental disorder of motor function, mixed specific developmental disorders, pervasive developmental disorders including childhood autism and Rett's syndrome and Asperger's syndrome. Disorders of intellectual development can include mild, moderate, and severe forms of mental retardation. Similarly, a number of behavioral and emotional disorders with onset usually occurring in childhood and adolescence, including hyperkinetic disorders, disturbances of activity and attention, conduct disorders, emotive disorders with onset specific to childhood, tic disorders including combined vocal and multiple motor tic disorder (e.g., de la Tourette), can be detected. It will be appreciated that this disorders listed herein are not exhaustive, and that the visualization system can be useful for additional mental disorders that are not listed herein.

The visualization system can also be used to detect and diagnose various diseases of the nervous system. Many of these diseases are listed in Block G (Chapter VI) of the ICD-1O. These diseases can include inflammatory diseases of the central nervous system, such as meningitis, encephalitis and abscesses, systemic atrophies primarily affecting the central nervous system, extrapyramidal and movement disorders, such as Parkinson's disease, and other diseases involving the cerebral cortex, and demyelinating diseases of the central nervous system, such as multiple sclerosis.

The system can also be applied in the detection and treatment of episodic and paroxysmal disorders. This includes the various forms of epilepsy, migraine, tension headache and other headache syndromes, not limited to cluster, transient cerebral ischemic attacks and related syndromes, such as Amaurosis fugax, vascular syndromes of brain in cerebrovascular diseases, sleep disorders including disorders of initiating and maintaining sleep (insomnias), disorders of excessive somnolence (hypersomnias), disruptions in circadian rhythm including jet lag, sleep apnea, narcolepsy, and cataplexy. In cerebralvascular disease, slowing of EEG frequencies is highly correlated with decreased regional blood flow. Cerebralvascular diseases include strokes, suspected strokes, or transient ischemic attacks.

The EEG visualization system can also be used to diagnose nerve and nerve root plexus disorder, as well as polyneuropathies and other disorders of the peripheral nervous system.

The system can further diagnose cerebral palsy and other paralytic syndromes, including infantile cerebral palsy, hemiplegia, paraplegia and triplegia where the cause is cortical in origin, as well as other disorders of the nervous system, including hydrocephalus, toxic encephalopathy, cerebral cysts, anoxic brain damage, benign intracranial hypertension, postviral fatigue syndrome, encephalopathy, unspecified compression of the brain, cerebral edema, and Reye's syndrome.

The EEG visualization system can also be applied in the diagnosis of other diseases and disorders involving the cerebral cortex, including many that are that are not explicitly mentioned above. These diseases can include disorders of belief and belief formation, such as delusions and delusional states, as delusional states have been found in some cases been found to involve low frequencies on the EEG. Cortical sensory disorders can also be detected, including visual disorders, such as cortical blindness and visual agnosia, acoustic disorders, such as cortical deafness and auditory agnosia, tactile disorders, disorders affecting the sense of smell, such as anosmia, vestibular disorders, such as vertigo, and visceral sensory disorders like irritable bowel syndrome and interstitial cystitis. The system can also be used to detect cortical damage, such as damage caused by stroke or brain injury. For example, it is possible to localize this damage using indicators of reduced cortical function in the damaged areas using the present invention. Head injuries have been associated in the medical literature with increased theta power, decreased delta power, decreased alpha power, low coherence, and increased asymmetry across the hemispheres of the brain. These abnormalities can be localized and better characterized using the present invention so as to provide diagnostic tests for the nature and severity of the injuries. Other space occupying lesions: This includes brain tumors and cysts that will likely have regions of reduced activity.

The visualization system can also be used in the diagnosis and treatment of chronic pain, for example, by measuring activity in cortical areas such as the anterior cingulate gyms. Chronic pain can include muscular and non-muscular pain, neuropathic pain, fibromyalgia and myofascial pain syndrome. Specific learning disorders can also be diagnoses, including disorders of the ability to acquire knowledge and, specifically, some specific disorders that have been associated with excess theta or decreased alpha and/or beta powers. The system can also diagnose disorders involving thought, feeling or combinations of the two, such as disorders of planning and foresight as well as of sentiments involving a combination of a thought and a feeling such guilt over an error, or the feeling of pride in an achievement.

The visualization system can also be used in the diagnosis and treatment of memory disorders, including disorders of memory storage and memory retrieval, reasoning disorders, including disturbances of making logical inferences, and evaluative disorders, including disorders involving the formation of evaluative judgments as to what the person deems to be good or bad. Similarly, the system can be applied to disorders of comprehension and understanding, such as agnosia, disorders of the self and the self-image, including disorder in self-representation and disorders of identity, and other circadian disorders affecting the cortex. Additional application of the system include detection and treatment of movement disorders, such as essential tremor and restless leg syndrome, social and conduct disorders, psychosomatic, speech and communication disorders, impulse control disorders, post traumatic stress disorder, and truth disorders, including any disorder in the brain of assigning an idea to the category of being true or untrue. It can also be used to diagnosis brain death.

The techniques and methods described herein can also be used for medical research and brain physiological research to understand the causes of diseases, human behavior, and mental processing, specifically as an aid to researching mental, psychological, and physical cortical processes and states. For example, the visualization technique can be used as an aid in the characterization of normal mental processes and normal physiological events and states, a tool in research into neural pathways and the discovery and further elucidation of migratory patterns of cortical electrical activity, and as an interpretation tool EEG recordings of normal and abnormal mental activity by revealing the sites of generators in the brain and the angular movements of electrical fields that contribute to EEG waveforms. Further, the ability to accurately trace the movement of current throughout the brain provided by the visualization system aids in the understanding of the translational and rotational movement of electrical fields produced by the brain as well as the recognition of functional elements of the brain, i.e. areas of the brain that work together to help perform a particular mental function. It will be appreciated that this research can aid in the characterization of a number of brain disorders, conditions, and states such as those listed previously so that effective diagnoses, monitoring methods and treatments can be developed.

The visualization system can also be used as an aid in the characterization of thoughts and ideas, feelings and emotions, beliefs, sensations, learning, understanding and comprehension, reasoning, desire and motivation, memory, evaluative processing, including processing of pleasure and pain, truth processing, planning, judgment, movement processing, speech and communication, representation, including self-representation, predispositions, and planning. Further, the system can be used in the process of drug development by helping determine the areas of the cerebral cortex where the electrical activity is affected by experimental and established pharmaceuticals, hence providing insight on the locations and mechanisms of action of these drugs.

Finally, the visualization system can be employed for non-medical purposes, such as games, entertainment, and industrial and mechanical applications. For example, the visualization and localization techniques could be used for training or controlling assistive devices. Alternatively, the system can be used to determine if a person is telling the truth or lying. Signature images and signature data patterns for lying and truthfulness may be identified through research trials utilizing the present invention. The trials may involve measuring people who are instructed to lie or instructed to tell the truth and who comply with this request while having their brain electrical activity recorded. The trial may also be conducted on people who actually lie when the person administering the test does not know during the testing session that the test subject is lying; this will capture cortical activity during actual lies. These two trials will provide a dataset of electrical activity of lying versus truthfulness and this dataset will later be used when testing future subjects for lying and will serve as a means for comparison. A conclusion that a patient has lied can be drawn if the examiner observes the display of a signature pattern for lying that is present in the database. Alternatively, it is possible to use statistical analysis of the data patterns to aid the examiner in identifying a lie.

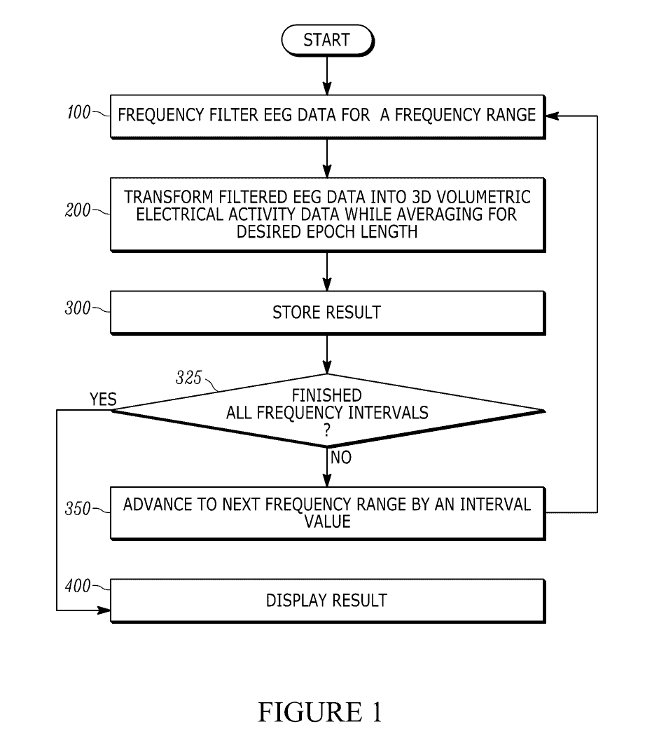

A general flowchart of a method in accordance with an aspect of the invention is depicted in FIG. 1. At step 100, EEG data is filtered to provide EEG data for a desired frequency range within a total range of frequencies. The EEG data can be filtered using a frequency filter algorithm such as a fast Fourier transform or windowed-sinc. The resulting EEG data then only contains frequencies ranging from the start to the end of that particular band.

In step 200, the 3-D electrical activity of the cerebral cortex is reconstructed by an inverse-solution approximation from the source EEG data into a 3-D-solution space comprising a plurality of voxels that define the regions of the brain occupied by the cerebral cortex. The 3-D-transformed EEG data is averaged for each region over a desired window of time, referred to as an epoch, to obtain a summary of the electrical activity for that epoch. By averaging the data, consistent activity within the brain is emphasized while minimizing the effect of transient activity that may appear throughout an EEG recording. The averages can be taken over any of several levels of detail, including voxels, Brodmann areas, minor anatomy areas called gyrii, and lobes. The averaged values themselves can represent the magnitude of electrical activity for each region, and/or the direction vectors for the electrical activity for each region.

At step 300, the averaged data is stored. The data can be stored in a large memory buffer, or provided directly to any sort of magnetic, optical, or flash-based storage. At step 325, it is determined if all desired frequency bands have been filtered, transformed and averaged. If so, the analysis is finished. Otherwise (N), the next frequency range is selected, according to a desired interval value, at step 350. For example, if a frequency range from four hertz to four hundred hertz is being analyzed in four-hertz increments, an eight to twelve hertz interval is selected immediately after a four to eight hertz interval has been processed. Once all frequency intervals have been processed, the results are then displayed at an associated display at step 400. For example, the activity in each of the plurality of voxels can be illustrated as a two-dimensional or three-dimensional image of all or a portion of the brain. An EEG generator is an electrical activity in the brain that is responsible for the waveforms seen on EEG. Source localization using inverse solutions may help to find generators. The visualization system can be used to help localize and isolate generators of interest from other generators in the brain. For example, the measured values can be evaluated to determine a frequency interval and a location associated with a given event seen in the raw EEG data.

As described previously, the measured activity can be used for any of a number of applications. For example, the visualization system can be used as a research tool to discover electrical biomarkers of brain states, or normal brain events, or diseased brain states or diseased brain events. A biomarker is an objective and measurable indicator of a pathogenic or physiologic (normal) biological process. A diagnostic biomarker is a biological marker that indicates the presence of a disease. It will be appreciated that the cortical activity produced by a system in accordance with present invention can be processed statistically to identify biomarkers from collected data. For the purposes of this document, the electrical activity occurring during making up a lie or lying is assumed to be a physiologically normal brain function. The system can be used to discover electrical biomarkers for events occurring in the brain while formulating a truthful expression or formulating a lie (i.e., biomarkers for lying and truth telling). For example, the system could be used to identify electrical biomarkers, which could be signature images and signature data patterns for lying and truthfulness, and the cortical activity of a subject can be measured after stimulating him or her with a question or other stimulus useful in stimulating his or her brain, such as showing the subject a murder weapon or other significant piece of evidence. The subject's reaction can be measured and compared to biomarkers found in an earlier research phase.

The system can be used to isolate electrical biomarkers of normal physiological events. For example, during sleep, the sleep spindle waveforms are considered to be an EEG biomarker for stage 2 sleep. The system can be used to make 2D and 3D images and paired histograms of the generators of these spindles. These biomarkers include average current density images over the duration of a sleep spindle for the specific frequency band of the spindle. The system can also be used on individuals to discover the presence or absence of known electrical biomarkers that were found during earlier research.

The data tables produced by the system can also be evaluated statistically for the purpose of diagnosis. For example, to diagnose a given disease, the cortical activity of a particular subject that has not been diagnosed can be measured compared to a database containing measurements from subjects having the disorder and/or to a normative database, including data from normal controls. If the subject's results are unlike the controls and like the subjects having the disorder, then the patient can be diagnosed with the disorder. This would be based on biomarkers for the disorder found during the research phase. For example, a biomarker for Alzheimer's might include reduced activity found in memory areas of the brain.

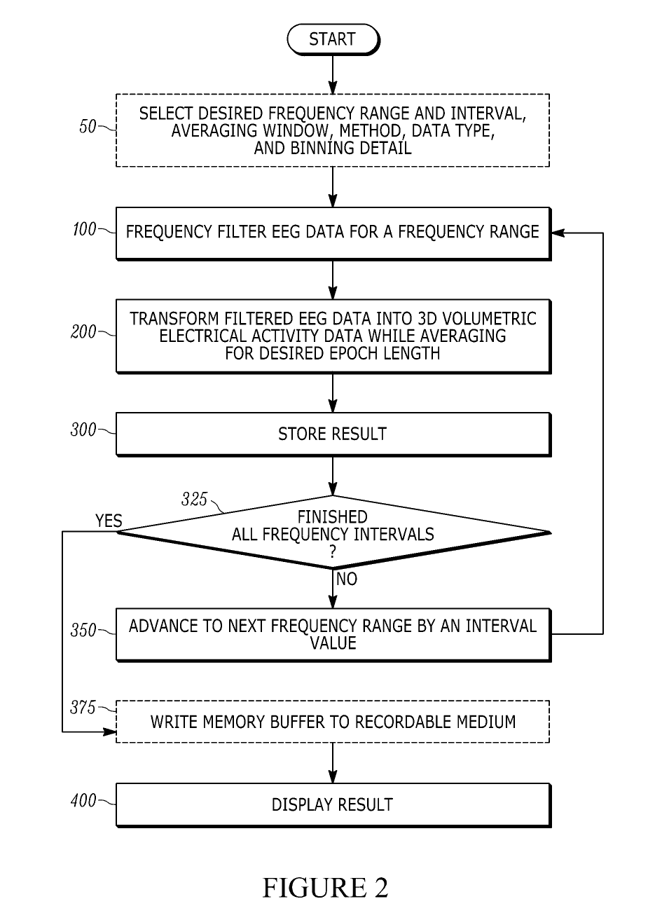

FIG. 2 depicts one implementation of a method in accordance with an aspect of the present invention. Steps 100, 200, 300, and 350 of FIG. 2 are similar to their corresponding steps in FIG. 1 and are not described again in the interest of brevity. The illustrated implementation utilizes a windowed-sinc filter for step 100, the LORETA algorithm in a 6239-voxel solution space based on the ICBM152 dataset for step 200, and stores the result in a large random access memory (RAM) buffer at step 300.

At step 50, each of a period of time representing a brain state or brain event, a desired frequency range, a frequency interval, an averaging window size, a method of averaging, and a level of binning detail are selected. The selection can be selected by a user at a user interface in a software implementation of the illustrated method. The desired frequency range is defined by selected lowest and highest frequencies to be analyzed—for example, 0-1024 Hz is an example of a desired frequency range.

The frequency interval defines the spacing and width of each frequency band within the desired frequency range. For example, with a spacing and width of 4 Hz would mean that 0-4 Hz, 4-8 Hz, 8-12 Hz, 12-16 Hz, . . . until 1020-1024 Hz would be examined within a desired range of 0 Hz to 1024 Hz. In some applications, the frequency bands will not be contiguous, such that the spacing of the frequency bands and the width as separate parameters. For example, where the frequency interval defines a spacing of 4 Hz, and width of 1 Hz, frequency bands of 0-1 Hz, 4-5 Hz, 8-9 Hz, 12-13 Hz, and so on until 1020-1021 Hz, would be analyzed.

The averaging window represents the length of data, measured in seconds or in frames with the number of frames is equal to the hardware sampling rate multiplied by however many seconds, to average in order to produce one data point. For example, if an averaging window of 3072 frames, or three seconds at a 1024 Hz sampling rate, were chosen, then for every 3072 frames in the EEG data, a single average number would be generated. If an EEG file consisted of 12000 frames, and the solution space consisted of 1000 voxels, then there would be 12,000,000 data points. With averaging, the four averaged data points would be generated for a particular region out of the 12000 frames, resulting in 4000 data points in total.

The illustrated method includes three methods by which averaging can be performed, although it will be appreciated that other methods can be utilized—a linear average, a “delta-sum” average, and a ‘Poisson’ average. A linear average is simply the arithmetic mean, determined as the sum of the values divided by the number of values. The “delta-sum” average represents the sum of the delta values divided by the number of values, where a delta value is the absolute value of the difference in current density value for one area from frame n-1 to frame n. Essentially, the delta-sum average represents an average change in the activity of a given region between subsequent frames of the data set. The ‘Poisson’ average keeps track of the region with the top electrical activity for each frame within a buffer the size of the solution space and then divides each value of the buffer by the averaging window size. For example, if voxel #23 had the highest activity 532 times within a 1000 frame window, and voxel #444 had the highest activity 231 times within the same window, the average values within the buffer after 1000 frames would be 0.532 for voxel #23 (523/1000) and 0.231 for voxel #444. The Poisson average provides an accessible way of quickly summarizing the regions of the brain experiencing heightened activity for a given epoch for a physician or researcher.

The data type is the type of data that is averaged, which can be either current densities or vectors. When EEG data is transformed into 3-D electrical activity by the inverse solution approximations, four quantities are produced for each voxel within the solution space: three vector components, representing X, Y, and Z components of the EEG data, and one scalar. The scalar quantity is the length of the 3-D vector and is known as the current density. Averaging of either quantity is possible with the above methodology.

Binning detail refers to the physical resolution, or level of detail of the analysis. If the averaging is not performed based on voxels, the smallest discrete unit of the measured data, then each averaging region consists of a list of voxels that comprises the region. The average electrical activity of the region is determined by the average values for the voxels comprising the region. At step 375, the final data is stored on a recordable computer readable medium. In the illustrated implementation, the recordable medium is a hard disk. The structure of the recorded data in the illustrated implementation is as follows:

Byte 0-4—number of data blocks (signed integer)

Bytes 4—end of file—a plurality of data blocks arranged sequentially, each as described below:

### A Single Data Block Structure

byte 0-4: method of averaging (signed integer)

byte 4-8: binning detail (signed integer)

byte 8-12: data type (signed integer)

byte 12-267: name of the data block (byte array [255])

byte 267-271: number of data points per frequency band (signed integer), denoted as dataSize

byte 271-275: low end of frequency range (floating point)

byte 275-279: high end of frequency range (floating point)

byte 279-283: increment between frequency bands (floating point)

byte 283-287: number of frequency bands examined in this data block (signed integer), denoted as numFreqs

byte 287-291: number of averaging windows (signed integer) denoted as epochs

byte 291-295: number of frames per averaging window (signed integer)

byte 295-299: number of variables per data point (current density=1, vectors=3; signed integer) denoted as nums

byte 299-299+size: the averaged data;
where size =dataSize * numFreqs *
epochs * nums;
(floating point array), arranged in a 4D array:
data[frequency band][data point][epoch][variable i. of data point]

byte 299+size-299+size*2 the standard deviations of the averaged data (same format as above; floating point array)

It will be appreciated that localization system and methods in accordance with the present invention provide an efficient method for summarizing EEG data for a human operator. In general, EEG data is somewhat opaque to a user, and significant processing is necessary to locate desired information from the returned signals. By automating the spectral analysis of the EEG data and representing average levels of neural activity in various regions across the brain, the data can be analyzed more generally, allowing for a general display of the measured neural activity. Accordingly, a user can readily identify portions of the brain responsible for given frequencies of neural activity even where such frequencies were not originally known to be of interest, greatly increasing the flexibility of the analysis.

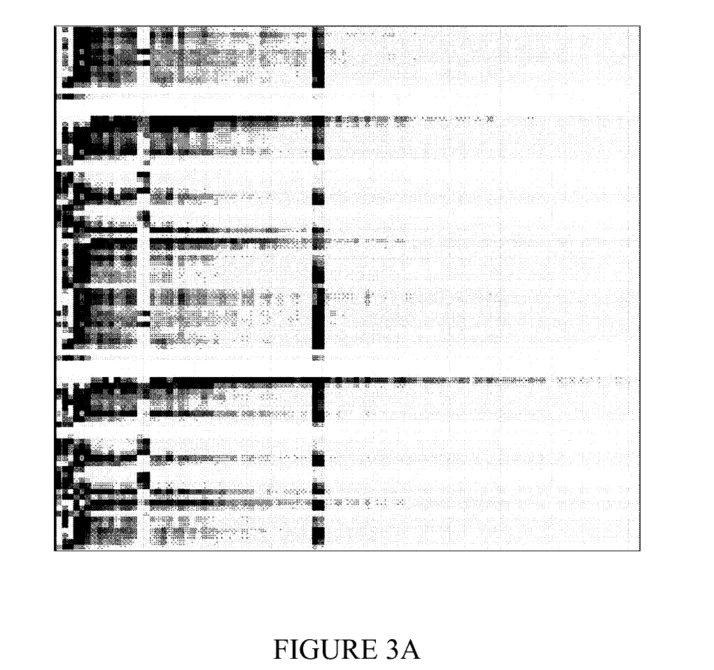

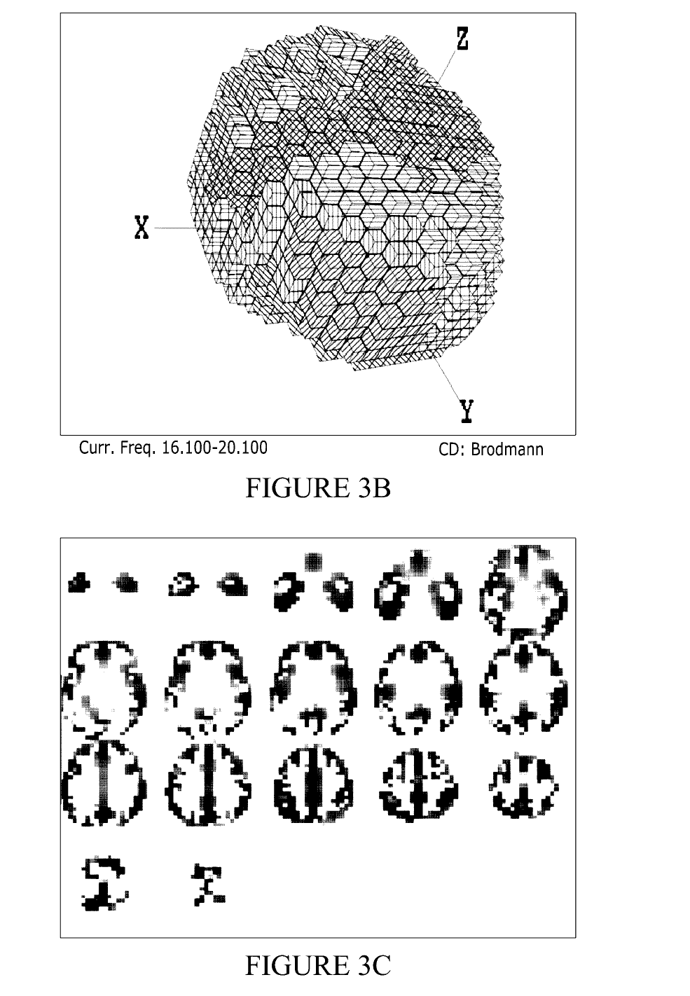

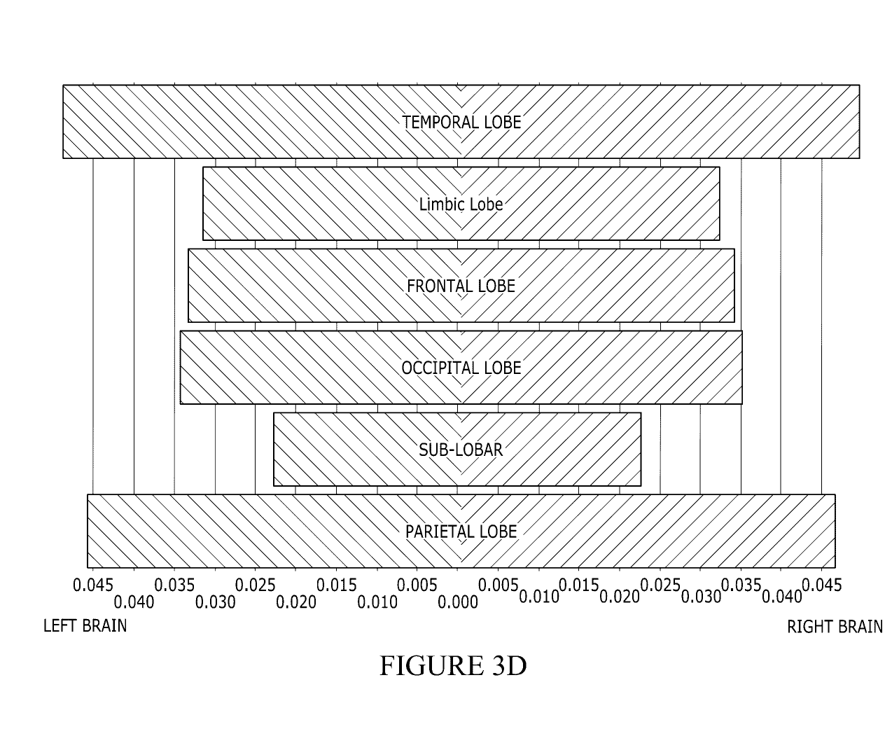

FIG. 3 depicts three exemplary methods by which the processed data can be visualized, utilized by the current reduction to practice. FIG. 3A depicts the entirety of the data in the form of a two-dimensional grid. The X-axis represents increasing frequency, and each square represents one frequency band. The example shown here is displaying one hundred frequency bands, starting at 0-4 Hz on the far left, to 396-400 Hz on the far right. The Y-axis represents the regions comprising the solution space. In the present example, left Brodmann area 1 is shown at the top, and right Brodmann Area 56 is shown at the bottom. The intensity of the square represents the magnitude of the electrical activity in this example. When displaying vector quantities, each square is further divided into three, displaying the magnitudes of each vector component.

FIG. 3B depicts the average current densities of a selected frequency band in three-dimensions based on the binning detail. The example shown here is displaying the average current densities of each Brodmann area for 12-16 Hz in 3-D. FIG. 3C depicts the average current densities of a selected frequency band in two-dimensional axial tomographic slices, based on the chosen binning detail. The bottom-most surface of the solution space is shown in top-left, and the top-most is at the bottom-right. Sagital and coronal axes are also possible. The example shown here depicts the same data as in FIG. 3B. FIG. 3D depicts the average current densities of a selected frequency band as a horizontal ‘paired histogram’, where the lengths of the horizontal bars correspond to the averaged current values of the area specified on the Y-axis. The portion of the bar that extends left of the y-axis represents areas within the left hemisphere of the cerebral cortex and the portion of the bar that extends right likewise represents areas on the right hemisphere. A final step (not shown) is the display of the aforementioned graphical information on a computer monitor.

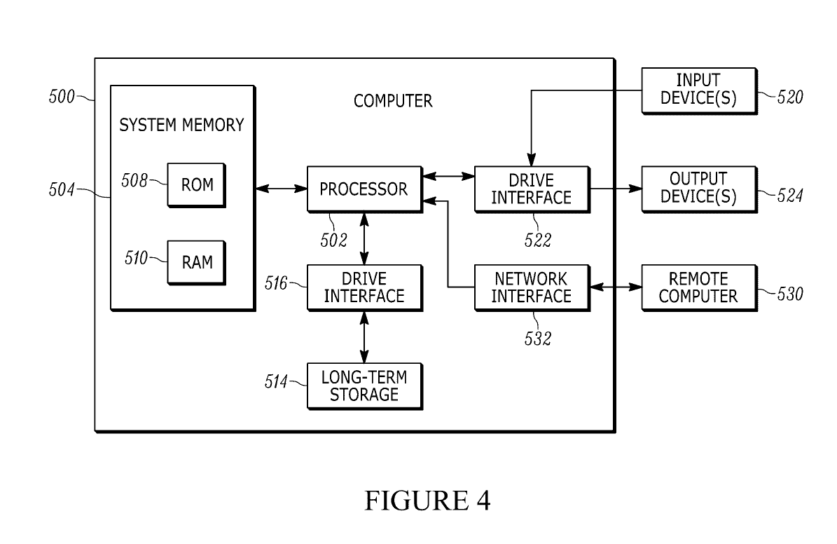

FIG. 4 illustrates a computer system 500 that can be employed to implement the systems and methods described herein as computer executable instructions, stored on a computer readable medium and running on the computer system. The computer system 500 can be implemented on one or more general purpose networked computer systems, embedded computer systems, routers, switches, server devices, client devices, various intermediate devices/nodes and/or stand alone computer systems. Additionally, the computer system 500 can be implemented as part of the computer-aided engineering (CAE) tool running computer executable instructions to perform a method as described herein.

The computer system 500 includes a processor 502 and a system memory 504. Dual microprocessors and other multi-processor architectures can also be utilized as the processor 502. The processor 502 and system memory 504 can be coupled by any of several types of bus structures, including a memory bus or memory controller, a peripheral bus, and a local bus using any of a variety of bus architectures. The system memory 504 includes read only memory (ROM) 508 and random access memory (RAM) 510. A basic input/output system (BIOS) can reside in the ROM 508, generally containing the basic routines that help to transfer information between elements within the computer system 500, such as a reset or power-up.

The computer system 500 can include one or more types of long-term data storage 514, including a hard disk drive, a magnetic disk drive, (e.g., to read from or write to a removable disk), and an optical disk drive, (e.g., for reading a CD-ROM or DVD disk or to read from or write to other optical media). The long-term data storage can be connected to the processor 502 by a drive interface 516. The long-term storage components 514 provide nonvolatile storage of data, data structures, and computer-executable instructions for the computer system 500. A number of program modules may also be stored in one or more of the drives as well as in the RAM 510, including an operating system, one or more application programs, other program modules, and program data.

A user may enter commands and information into the computer system 500 through one or more input devices 520, such as a keyboard or a pointing device (e.g., a mouse). Further, the computer system 500 can receive data from one or more sensors, such as conductive leads for an EEG system. These and other input devices are often connected to the processor 502 through a device interface 522. For example, the input devices can be connected to the system bus by one or more a parallel port, a serial port or a universal serial bus (USB). One or more output device(s) 524, such as a visual display device or printer, can also be connected to the processor 502 via the device interface 522.

The computer system 500 may operate in a networked environment using logical connections (e.g., a local area network (LAN) or wide area network (WAN) to one or more remote computers 530. A given remote computer 530 may be a workstation, a computer system, a router, a peer device or other common network node, and typically includes many or all of the elements described relative to the computer system 500. The computer system 500 can communicate with the remote computers 530 via a network interface 532, such as a wired or wireless network interface card or modem. In a networked environment, application programs and program data depicted relative to the computer system 500, or portions thereof, may be stored in memory associated with the remote computers 530.

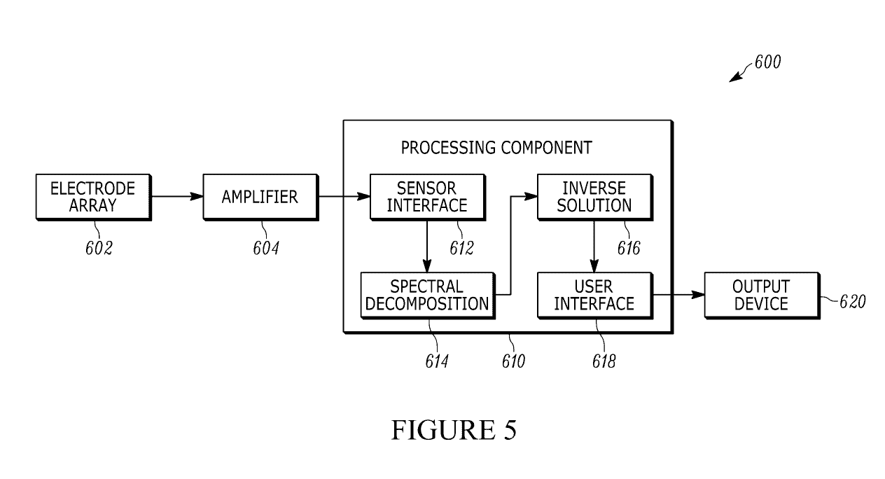

FIG. 5 illustrates one implementation of a system 600 in accordance with an aspect of the present invention. The system 600 includes an electrode array 602 configured to take measurements of electrical potential in a region on interest, such as along the scalp of a patient. The measurements from the electrode array 602 are amplified at an amplifier 604, and provided to a data processing apparatus 610. It will be appreciated that the data processing apparatus can be implemented as software running on a general purpose computer, as dedicated hardware, or as some combination of dedicated hardware and an appropriately programmed general purpose computer.

The data processing apparatus 610 comprises a spectral decomposition component 614 configured to filter the EEG data contained within a plurality of channels into desired frequency subranges within a total range of frequencies. The EEG data is divided using a frequency filter algorithm such as a fast Fourier transform or windowed-sinc. Each EEG data channel then only contains frequencies ranging from the start to the end of that particular band.

An inverse solution component 616 can apply an inverse-solution approximation to reconstruct the 3-D electrical activity of the cerebral cortex from the source EEG data within a given channel into a 3-D solution space consisting of voxels that define the regions of the brain occupied by the cerebral cortex. The 3-D-transformed EEG data is simultaneously averaged for each region over the desired window of time (epoch) to obtain a summary of the electrical activity, or in other words, the “brain state”. Averaging highlights consistent activities while reducing the transient activity that may appear throughout a recording. The available levels of detail include averaging based on voxels, Brodmann areas, minor anatomy areas called gyrii, and lobes. The values themselves can represent the magnitude of electrical activity for each region, and/or the direction vectors for the electrical activity for each region. The constructed 3-D data can then be provided to a user interface 618 for display at an associated output device 620, such as video monitor or printer. For example, the output can include color-coded images of the 3-D data for all or a portion of the cortex, datasets giving raw values or average values for individual voxels, Brodmann areas, gyrii, or lobes, or additional graphical representations of these values. The user interface 618 can be configured to allow the user to select among a plurality of visualization options, such that the display can be adapted to various applications.

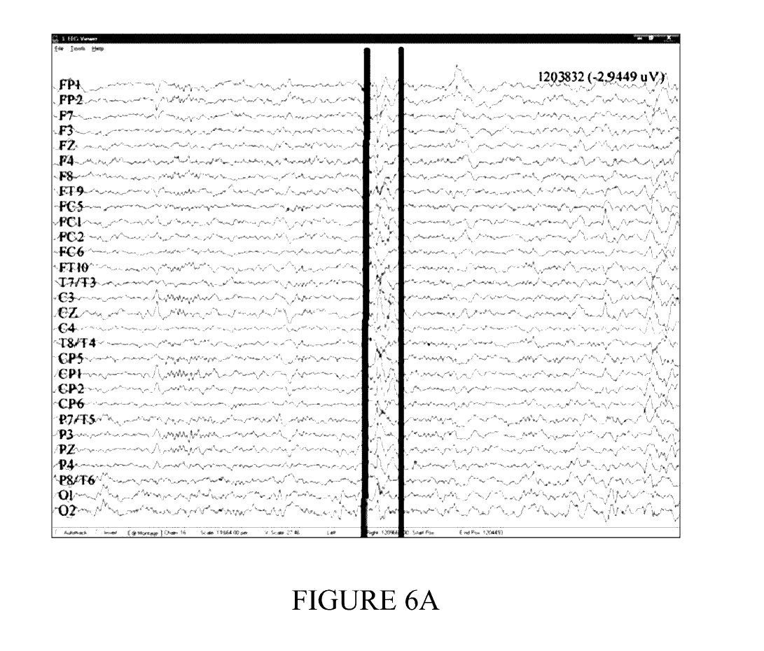

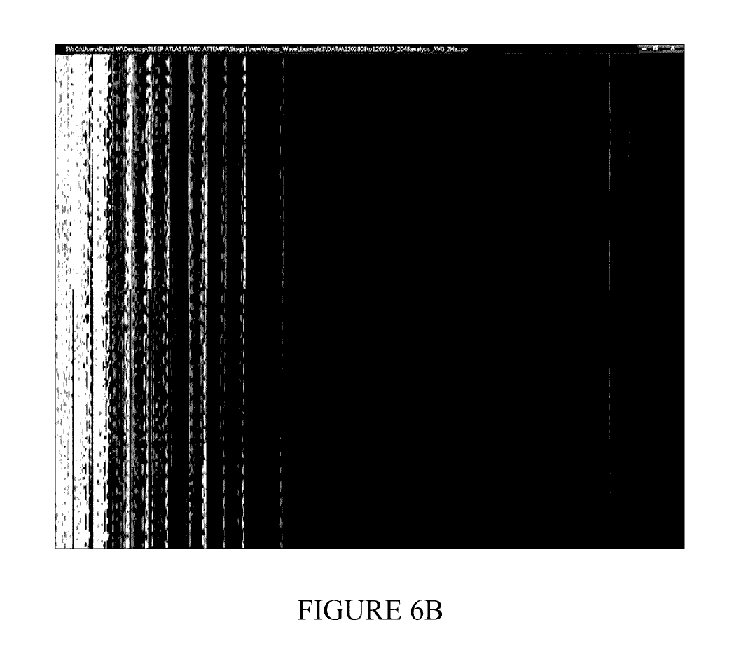

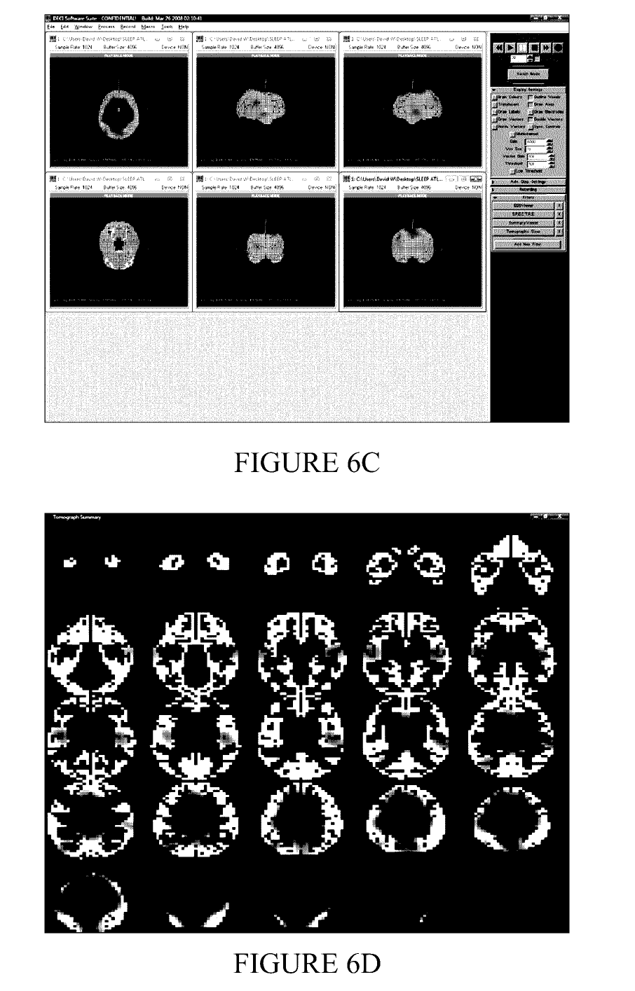

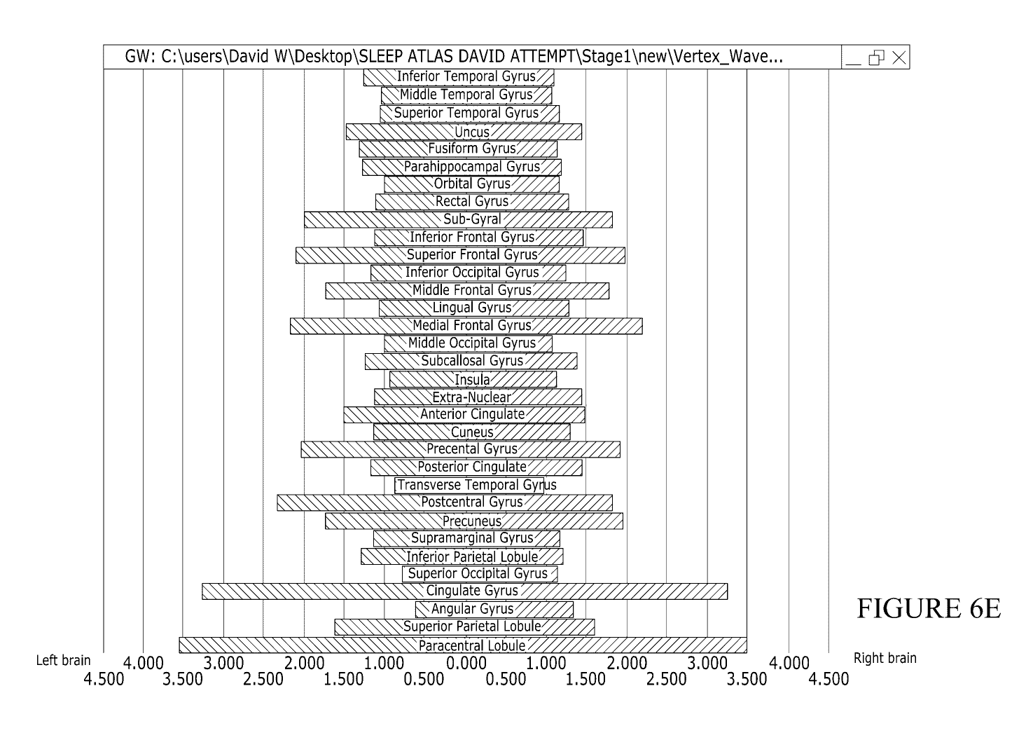

FIG. 6 depicts a series of images (6A-6e) which combined serve as an example of how to use the linear average functionality in the visualization system to find a summary of the generator activity of an EEG waveform. One must identify a waveform of interest. In this instance, it is a vertex waveform in the brain of a sleeping healthy young man from stage one sleep. FIG. 6A depicts an EEG showing a waveform of interest which is a vertex waveform (i.e., vertex wave) just after the dark vertical line near the middle of this EEG. It appears as the sudden onset of complex groups of hills and valleys in all the electrodes occupying about two-thirds of the sixth segment from the left of the page of the EEG in 6A. A generator is causing hills and valleys seen in all these electrodes (which are listed at the far left). The tallest hill is in the Cz electrode. To find the generator responsible for this series of shapes, the first step is to “cut” out the segment of interest from the EEG containing only this waveform.

FIG. 6B shows 2-D images created by the visualization system. From these images, it is clear, especially viewed in colour, that the strongest activity is in the first three bands from the left. When viewed in colour, the heavy red pixilation indicating strong activity. The operator can then select a frequency sub-band. The third band from the left is the strongest. In this case, it is the 4-6 Hz sub-band. FIG. 6C shows six 3-D views of the surface of the brain for the linear averaged activity of the vertex wave for the 4-6 Hz sub-band. By inspection of these six views, it is apparent to one aware of the anatomy of the cortex that the generator is coming from the top of the brain. FIG. 6D shows axial tomography of the same vertex wave epoch and it confirms that the neural generators for this vertex wave are in the upper and midline regions of the brain. For example, the fifth row of images, approaching the vertex of the brain, shows a diffuse pattern of symmetrical activation. FIG. 6E demonstrates how the system helps to provide the anatomical names for generators of the vertex wave. It shows that the strongest activity for this generator for the 4-6 Hz sub-band is in the left and right paracentral lobules and the left and right cingulate gyrii.

The present invention should not be considered limited to the particular examples described above, but rather should be understood to cover aspects of the invention as fairly set out in the attached claims. Various modifications, equivalent processes as well as numerous structures to which the present invention may be applicable will be readily apparent to those of skill in the art to which the present invention is directed upon review of the specifications.

1-20. (canceled)

21. A method for the analysis of raw electroencephalographic (EEG) data of a subject representing one of a brain event and a brain state comprising:
generating the raw EEG data via an electrode array;
selecting a period of time representing the one of a brain event and a brain state and an averaging window at a user interface;
selecting a first frequency interval from the plurality of frequency intervals;
iteratively performing the following steps on a subset of the RAW EEG data representing the selected period of time until each of a plurality of frequency intervals have been evaluated:
filtering the subset of the raw EEG data to produce a filtered EEG signal representing data within the selected frequency interval;
transforming the data represented by the filtered EEG signal over a plurality of frames of EEG data via an inverse solution approximation algorithm as to determine, for the selected frequency interval, respective sets of values at a plurality of locations within a brain of the subject, each value representing a current density within the frequency interval associated with the frequency interval at a corresponding location;
averaging the sets of values for each location of the plurality of locations for the selected frequency interval over the selected averaging window within the selected period of time to produce, for each frequency interval, at least one averaged value for each of the plurality of locations within the brain of the subject; and
advance to a next frequency interval from the plurality of frequency intervals; and
storing the at least one averaged value for each of the plurality of frequency intervals on a non-transitory computer as a set of averaged values.

22. The method of claim 21, wherein averaging the values representing the current density comprises:
determining a location of the plurality of locations having a maximum value of current density for a given data frame of a plurality of data frames comprising a given epoch; and
determining the averaged value for each location as a number of frames for which the location had the maximum value of current density divided by the number of data frames in the plurality of data frames comprising the epoch.

23. The method of claim 21, further comprising displaying the set of averaged values to a user at an output device.

24. The method of claim 23, wherein displaying the set of averaged values comprises displaying a paired histogram in which each of a plurality of histogram bars on a first side of an axis represent the averaged current values of one of the plurality of locations within a left hemisphere of the brain and each of a plurality of histogram bars on a second side of the axis represent the averaged current values of a corresponding plurality of locations within a right hemisphere of the brain.

25. The method of claim 21, wherein the subject is a first subject of a plurality of subjects and the plurality of averaged values is a first plurality of averaged values and further comprising:
analyzing a second subject to determine a second plurality of averaged values corresponding to the plurality of locations within the brain of the second subject, the first subject and the second subject sharing a clinically relevant characteristic; and
combining the first plurality of averaged values and the second plurality of averaged values to generate a normative dataset, suitable for medical and psychological research, representing the clinically relevant characteristic.

26. The method of claim 25, the clinically relevant characteristic comprising at least one of a physiological state and a physiological event that are associated with a healthy brain and further comprising performing a statistical analysis on the normative dataset to identify a biomarker associated with the at least one physiological state or event.

27. The method of claim 25, the further comprising performing a statistical comparison of a dataset representing a disease of interest to the normative dataset to identify a biomarker associated with the disease.

28. The method of claim 21, further comprising subjecting the subject to a stimulus, wherein selecting the period of time representing the one of a brain event or a brain state comprises selecting the period of time to represent a response to the stimulus.

29. The method of claim 21, further comprising comparing the plurality of averaged values to one of a dataset representing truthfulness and a dataset representing falsehood to evaluate the truthfulness of a response of the subject.

30. The method of claim 21, comparing the plurality of averaged values to one of a normative database and a disease database to locate a disease biomarker.

31. The method of claim 21, wherein averaging the sets of values for each location of the plurality of locations for the selected frequency interval over the selected averaging window comprises determining one of the arithmetic mean of a subset of the set of values, the arithmetic mean of a plurality of delta values determined from the subset of the set of values subset of the set of values, each of the subset of the set of values representing the electrical activity within the selected frequency interval at the given location for a corresponding data frame within the averaging window, and a delta value for a location of the plurality of locations is the absolute value of a difference in current density at the location from a first frame to a second, consecutive frame.

32. A system for the analysis of raw electroencephalographic (EEG) data of a subject representing one of a brain event and a brain state comprising:
an electrode array that takes measurements of electrical potential as raw electroencephalographic (EEG) data;
a user interface that allows a user to select a plurality of contiguous frequency intervals and a period of time representing the one of a brain event and a brain state;
a data processing component that determines, for a subset of the raw EEG data representing the selected period of time, a spatial mapping of electrical activity for each of the plurality of frequency intervals, the data processing component comprising:
a spectral decomposition component that selects a frequency interval of the plurality of frequency intervals and filters the subset of the raw EEG data to provide an EEG data set containing data only within the selected frequency interval; and
an inverse solution component that transforms the EEG data set containing data only within the selected frequency interval into a spatial mapping of electrical activity so as to provide a set of parameters, each parameter representing an average electrical activity at an associated location within the brain over an epoch within the period of time;
wherein the spectral decomposition component and the inverse solution component sequentially select each of the plurality of frequency intervals and provide a corresponding set of parameters for each frequency interval; and
a non-transitory storage medium that stores the set of parameters for each of the plurality of frequency intervals.

33. The system of claim 32, further comprising an output device that displays at least one of the sets of parameters stored on the non-transitory storage medium.

34. The system of claim 33, wherein the output device displays at least two of the sets of parameters stored on the non-transitory storage medium.

35. The system of claim 34, the output device providing a two-dimensional grid having a plurality of pixels, with each column representing one of the plurality of frequency bands and each row representing a location of the plurality of locations.

36. The system of claim 35, the output device displaying the set of parameters such that the degree of electrical activity at each location is signified via a color of a graphic representing the location.

37. The system of claim 33, the output device displaying the set of parameters for given frequency band of the plurality of locations as a paired histogram, wherein a length of each bar to a left side of a vertical axis represents an averaged value for a given location within a left hemisphere of a cerebral cortex of the subject and a length of each bar to a right side of the vertical axis represents a given location within a right hemisphere of the cerebral cortex of the subject.

38. The system of claim 32, the plurality of locations comprising voxels within a three-dimensional representation of the brain.

39. The system of claim 32, wherein the inverse solution component provides the parameter associated with a given location of the plurality of locations as one of the arithmetic mean of a plurality of values and the arithmetic mean of a plurality of delta values determined from the plurality of values, each of the plurality of values representing the electrical activity within the selected frequency interval at the given location for a corresponding data frame within the epoch, and a delta value for a location of the plurality of locations is the absolute value of a difference in current density at the location from a first frame to a second, consecutive frame.

40. A method for the analysis of raw electroencephalographic (EEG) data of a subject representing one of a brain event and a brain state comprising:
generating the raw EEG data via an electrode array;
selecting a period of time representing the one of a brain event and a brain state and an averaging window at a user interface;
selecting a first frequency interval from the plurality of frequency intervals;
iteratively performing the following steps on a subset of the RAW EEG data representing the selected period of time until each of a plurality of frequency intervals have been evaluated:
filtering the subset of the raw EEG data to produce a filtered EEG signal representing data within the selected frequency interval;
transforming the data represented by the filtered EEG signal over a plurality of frames of EEG data via an inverse solution approximation algorithm as to determine, for the selected frequency interval, respective sets of values at a plurality of locations within a brain of the subject, each value representing a current density within the frequency interval associated with the frequency interval at a corresponding location;
averaging the sets of values for each location values corresponding to the current density associated with the selected frequency interval over the selected averaging window within the selected period of time to produce, for each frequency interval, at least one averaged value for each of the plurality of locations within the brain of the subject; and
advance to a next frequency interval from the plurality of frequency intervals;
storing the at least one averaged value for each of the plurality of frequency intervals on a non-transitory computer as a set of averaged values; and
displaying the set of averaged values to a user at an output device.
